(19) United States
(12) Patent Application Publication
(10) Pub. No.: US 2024/0158342 A1
(43) Pub. Date: May 16, 2024

(54) PURIFICATION OF ALIPHATIC TAURATE AMIDE

(71) Applicant: SPECIALTY OPERATIONS FRANCE, Lyon (FR)

(72) Inventors: Hocine KABIR, 34 rue de la Source (FR); Julien RACHET, Vernaison (FR); Sébastien FRIDIERE, LUCENAY (FR); Damien BOURCIER, Saint-Priest (FR)

(21) Appl. No.: 18/551,008

(22) PCT Filed: Mar. 3, 2022
(86) PCT No.: PCT/EP2022/055477

§ 371 Date: Sep. 19, 2023

(30) Foreign Application Priority Data
Mar. 19, 2021 (EP) 21163733.5
Dec. 21, 2021 (EP) 20216191.3

(51) Int. Cl. C07C 303/44; C07C 303/02

(52) U.S. Cl. CPC C07C 303/44; C07C 303/02

(57) ABSTRACT

The present invention relates to the purification of aliphatic taurate amides by liquid-liquid extraction, notably to remove free fatty carboxylic acids. Such aliphatic taurate amides may notably be obtained by reaction of a carboxylic acid with alkali metal salt of N-methyl taurine.

The present invention relates to the purification of aliphatic taurate amides by liquid-liquid extraction, notably to remove free fatty carboxylic acids. Such aliphatic taurate amides may notably be obtained by reaction of a carboxylic acid with alkali metal salt of N-methyl taurine.

This application claims priorities filed on 19 Mar. 2021 in Europe with Nr 21163733.5 and 21 Dec. 2021 in Europe with Nr 21216191.3 (the whole content of each of these applications being incorporated herein by reference for all purposes).

### PRIOR ART

Aliphatic taurate amides are anionic surfactants offering excellent foaming properties for various applications, notably with luxurious skin feel, mildness and lathering properties. Aliphatic taurate amides help to build the viscosity in sulfate-free chassis answering consumer request of having a mild and respectful formulations in cosmetic applications.

Production of aliphatic taurate amides made from an amidation reaction of taurine or salts of taurine with fatty acid, such as C₆ to C₂₄ chain length fatty acid is well known. However, said reaction is not necessarily complete and led to the presence of residual amount of fatty acids that needs to be removed by separation methods for instance by a treatment with an inorganic or organic base.

Several prior art documents refers to such a separation method, notably by distillation or crystallization but there is a need for a simple and industrial purification process to remove fatty acids for the production of high quality aliphatic taurate amides.

### INVENTION

The present invention aims at solving this technical problem and other non-addressed issues. Indeed, it appears that the use of ethyl acetate can be efficiently used as solvent for a liquid/liquid extraction to remove free fatty acids from a composition comprising at least aliphatic taurate amide and fatty acids, notably aliphatic taurate amide obtained by reaction of a C₆-C₂₄ carboxylic acid with alkali metal salt of N-methyl taurine, preferably alkyl taurate amide obtained by reaction of a C₆-C₂₄ carboxylic acid with alkali metal salt of N-methyl taurine.

The present invention refers then to a process of purifying a crude aliphatic taurate amide composition comprising at least the step of proceeding with a liquid/liquid extraction with ethyl acetate of an aqueous composition comprising at least aliphatic taurate amide and C₆-C₂₄ carboxylic acid.

The present invention specifically refers to a process of purifying a crude alkyl taurate amide composition comprising at least the step of proceeding with a liquid/liquid extraction with ethyl acetate of an aqueous composition comprising at least alkyl taurate amide and C₆-C₂₄ carboxylic acid.

The present invention also refers to a process for purifying aliphatic taurate amide from a crude aliphatic taurate amide composition comprising at least the step of proceeding with a liquid/liquid extraction with ethyl acetate of an aqueous composition comprising at least aliphatic taurate amide and C₆-C₂₄ carboxylic acid; preferably a process for purifying alkyle taurate amide from a crude alkyle taurate amide composition comprising at least the step of proceeding with a liquid/liquid extraction with ethyl acetate of an aqueous composition comprising at least alkyle taurate amide and C₆-C₂₄ carboxylic acid.

Crude aliphatic taurate amide composition preferably refers to a composition comprising aliphatic taurate amide, preferably obtained by reaction of a C₆-C₂₄ carboxylic acid with alkali metal salt of N-methyl taurine. Said crude aliphatic taurate amide composition usually comprises aliphatic taurate amide, alkali metal salt of N-methyl taurine, and C₆-C₂₄ carboxylic acid (or free fatty acids).

Such a purification process permits the production of high quality aliphatic taurate amides by notably removing C₆-C₂₄ carboxylic acids and a composition comprising aliphatic taurate amides with a low content of C₆-C₂₄ carboxylic acids. Such a process is simple, rapid and complete for making aliphatic taurate amides having minimal operating conditions. This improved process allows production of such taurate amides at lower reaction temperatures and/or higher yields and/or with decreased formation of colored byproducts. This process then allows purification of aliphatic taurate amides with reaction conditions sufficiently mild to avoid degradation of aliphatic taurate amides.

This process is more economical and/or more readily controlled and/or more independent of impurities in the starting materials, without producing any toxic or highly undesirable products.

The invention also concerns a purified product susceptible to be obtained by the above-mentioned process.

The invention also refers to the use of ethyl acetate as solvent for liquid/liquid extraction of an aqueous composition comprising an aliphatic taurate amide, notably an aqueous composition comprising at least aliphatic taurate amide and C₆-C₂₄ carboxylic acid; aliphatics taurate amide being preferably obtained by reaction of a C₆-C₂₄ carboxylic acid with alkali metal salt of N-methyl taurine.

The present invention also refers to a composition, notably obtained by the process of the invention, comprising at least:
a) aliphatic taurate amide; preferably alkyl taurate amide;
b) C₆-C₂₄ carboxylic acid; and
c) ethyl acetate.

The present invention also refers to a liquid composition, notably obtained by the process of the invention, comprising at least, preferably a composition consisting of:
a) from 10 to 50% by weight of aliphatic taurate amide, preferably alkyl taurate amide;
b) from 0 to 2% by weight of C₆-C₂₄ carboxylic acid;
c) from 0 to 2% by weight of inorganic salt;
d) from 0 to 2% by weight of alkali metal salt of N-methyl taurine;
e) from 0 to 0.2% by weight of ethyl acetate; and
f) water the proportion by weight are calculated with respect to the total weight of the composition.

The present invention also refers to a solid composition, notably obtained by the process of the invention, comprising at least, preferably a composition consisting of:
a) from 80 to 99% by weight of aliphatic taurate amide, preferably alkyl taurate amide;
b) from 0 to 5% by weight of C₆-C₂₄ carboxylic acid;
c) from 0 to 2% by weight of inorganic salt;
d) from 0 to 2% by weight of alkali metal salt of N-methyl taurine;
e) from 0 to 0.1% by weight of ethyl acetate; and
f) from 0 to 10% by weight of water
the proportion by weight are calculated with respect to the total weight of the composition.

### DETAILED INVENTION

### Definitions

Should the disclosure of any patents, patent applications, and publications which are incorporated herein by reference conflict with the description of the present application to the extent that it may render a term unclear, the present description shall take precedence.

While the following terms are believed to be understood by one of ordinary skill in the art, the following definitions are set forth to facilitate explanation of the presently disclosed subject matter. Unless defined otherwise, all technical and scientific terms used herein have the same meaning as commonly understood by one of ordinary skill in the art to which the presently disclosed subject matter pertains. Although any methods, devices, and materials similar or equivalent to those described herein can be used in the practice or testing of the presently disclosed subject matter, representative methods, device, and materials are now described.

Should the disclosure of any patents, patent applications, and publications which are incorporated herein by reference conflict with the description of the present application to the extent that it may render a term unclear, the present description shall take precedence.

Throughout this specification, unless the context requires otherwise, the word “comprise” or “include”, or variations such as “comprises”, “comprising”, “includes”, including” will be understood to imply the inclusion of a stated element or method step or group of elements or method steps, but not the exclusion of any other element or method step or group of elements or method steps. According to preferred embodiments, the word “comprise” and “include”, and their variations mean “consist exclusively of”.

As used in this specification, the singular forms “a”, “an” and “the” include plural aspects unless the context clearly dictates otherwise. The term “and/or” includes the meanings “and”, “or” and also all the other possible combinations of the elements connected to this term.

The term “between” should be understood as being inclusive of the limits.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a temperature range of about 120° C. to about 150° C. should be interpreted to include not only the explicitly recited limits of about 120° C. to about 150° C., but also to include sub-ranges, such as 125° C. to 145° C., 130° C. to 150° C., and so forth, as well as individual amounts, including fractional amounts, within the specified ranges, such as 122.2° C., 140.6° C., and 141.3° C., for example.

The term “aryl” refers to an aromatic carbocyclic group of 6 to 18 carbon atoms having a single ring (e.g. phenyl) or multiple rings (e.g. biphenyl), or multiple condensed (fused) rings (e.g. naphthyl or anthranyl). Aryl groups may also be fused or bridged with alicyclic or heterocyclic rings that are not aromatic so as to form a polycycle, such as tetralin. The term “aryl” embraces aromatic radicals such as phenyl, naphthyl, tetrahydronaphthyl, indane and biphenyl. An “arylene” group is a divalent analog of an aryl group.

The term “heteroaryl” refers to an aromatic cyclic group having 3 to 10 carbon atoms and having heteroatoms selected from oxygen, nitrogen and sulfur within at least one ring (if there is more than one ring).

The term “aliphatic group” includes organic moieties characterized by straight or branched-chains, typically having between 1 and 18 carbon atoms.

The term “aliphatics” refers to substituted or unsubstituted saturated alkyl chain having from 1 to 18 carbon atoms, substituted or unsubstituted alkenyl chain having from 1 to 18 carbon atoms, substituted or unsubstituted alkynyl chain having from 1 to 18 carbon atoms. In complex structures, the chains may be branched, bridged, or cross-linked. Aliphatic groups include alkyl groups, alkenyl groups, and alkynyl groups.

As used herein, “alkyl” groups include saturated hydrocarbons having one or more carbon atoms, including straight-chain alkyl groups, such as methyl, ethyl, propyl, butyl, pentyl, hexyl, heptyl, octyl, nonyl, decyl, cyclic alkyl groups (or “cycloalkyl” or “alicyclic” or “carbocyclic” groups), such as cyclopropyl, cyclopentyl, cyclohexyl, cycloheptyl, and cyclooctyl, branched-chain alkyl groups, such as isopropyl, tert-butyl, sec-butyl, and isobutyl, and alkyl-substituted alkyl groups, such as alkyl-substituted cycloalkyl groups and cycloalkyl-substituted alkyl groups.

As used herein, “alkenyl” or “alkenyl group” refers to an aliphatic hydrocarbon radical which can be straight or branched, containing at least one carbon-carbon double bond. Examples of alkenyl groups include, but are not limited to, ethenyl, propenyl, n-butenyl, i-butenyl, 3-methylbut-2-enyl, n-pentenyl, heptenyl, octenyl, decenyl, and the like. The term “alkynyl” refers to straight or branched chain hydrocarbon groups having at least one triple carbon to carbon bond, such as ethynyl.

The term “arylaliphatics” refers to an aryl group covalently linked to an aliphatics, where aryl and aliphatics are defined herein.

As used herein, the terminology “(Cn-Cm)” in reference to an organic group, wherein n and m are each integers, indicates that the group may contain from n carbon atoms to m carbon atoms per group.

Alkyl Taurate Amide and Production Processes

Taurates (or taurides) are a group of anionic surfactants. They are composed of a hydrophilic head group, consisting of N-methyltaurine (2-methylaminoethanesulfonic acid) and a lipophilic residue, consisting of a long-chain carboxylic acid (fatty acid), both linked via an amide bond. Aliphatic taurate amides of the invention are salts, such as salts of alkali metals.

Aliphatic taurate amide may be for instance alkyl taurate amide or alkenyl taurate amides.

Aliphatic taurate amides of the invention may be obtained by various processes, notably by involving the reaction of a C₆-C₂₄ carboxylic acid with alkali metal salt of N-methyl taurine. Aliphatic taurate amides as obtained with such a process are often called crude aliphatic taurate amides. Aliphatic taurate amides are preferably obtained by reaction of a C₆-C₂₄ carboxylic acid with alkali metal salt of N-methyl taurine.

Alkali metal salt may be for instance lithium (Li), sodium (Na), and potassium (K), preferably sodium.

Aliphatic taurate amides may be chosen in the group consisting of: sodium methyl lauroyl taurate, sodium methyl ceteoyl taurate, sodium methyl palmitoyl taurate, sodium methyl oleyl taurate, sodium methyl stearoyl taurate, and sodium methyl cocoyl taurate.

Any C₆-C₂₄ carboxylic acid or fatty acid may be employed in the process of this invention, preferably C₈ to C₂₂ or C₈ to C₂₀, carboxylic acids. The acid may be derived from a saturated or unsaturated aliphatic, alicyclic or aliphatic aromatic acid.

C₆-C₂₄ carboxylic acids are preferably chosen in the group consisting of: caprylic acid, octanoic acid, decanoic acid, lauric acid, cocoyl acid, tridecylic acid, myristic acid, pentadecylic acid, palmitic acid, margaric acid, stearic acid, nonadecylic acid, arachidic acid, behenic acid, pyroterebic acid (4-methyl-β-pentenoic acid), α-ethylcrotonic acid, teracrylic acid, d-citronellic acid, θ-undecylenic acid, oleic acid, elaidic acid, erucic acid, sorbic acid, stearolic acid, linoleic acid, behenoleic acid, ricinoleic acid, margaric acid, arachidic acid and the like.

Non-limiting examples of unsaturated fatty acids include palmitoleic acid, oleic acid, octadecenoic acid, linoleic acid, gamma-linoleic acid, alpha linoleic acid, arachidic acid, eicosenoic acid, homogamma linoleic acid, arachidonic acid, eicosapenenoic acid, docosadienoic acid, heneicosapentaenoic, docosatetraenoic acid.

In addition to these acids, acids obtained from tall oil, hydrogenated tall oil, hydrogenated tallow, and the like may be employed. Acid mixtures from various natural plant and animal oils, such as olive, tallow, castor, peanut, coconut, soybean, cottonseed, linseed, palm, corn, and the like may also be employed.

Coco fatty acids are preferred, the coconut fatty acids typically being a mixture of C₁₂ carboxylic acids in the highest proportion with lower proportions of C₁₄ carboxylic acids, and still lower proportions of acids of lower and higher carbon content, mostly saturated.

A blend of C₆ to C₂₀ carboxylic acids may also be used in the reaction. A blend of C₁₂ to C₂₀ carboxylic acids may also be used in the reaction. Both are coco fatty acid: C₆-C₂₀ is the full cut coco fatty acid while the C₁₂-C₂₀ is the topped (or hardened) coconut fatty acid A typical composition of a full cut fatty acid is as follow (% by weight): C₆: <1%; C₈: 4-10%; C₁₀: 4-8%; C₁₂: 45-54%; C₁₄: 15-21%; C₁₆: 7-13%; C₁₈+C_{18:1}: 6-14% and C₂₀: <0.2%. A typical composition of a topped coco fatty acid is as follow (% by weight): C₈: <1%; C₁₀: <1%; C₁₂: 45-60%; C₁₄: 17-27%; C₁₆: 5-15%; C₁₈+C_{18:1}<17%, and C₂₀: <0.5%.

Molar ratio of total fatty acid to salt of N-methyl taurine, both total fatty acid and salt measured as anhydrous basis, may be comprised between 1:1 and 5:1, preferably 1.1:1 to 1.5:1. If more than one fatty acid is used, the ratio is defined as the molar ratio of all fatty acid in the mixture to taurine salt.

Reaction temperature for production of aliphatic taurate amides may be comprised from 150 to 300° C., preferably from 200 to 250° C. Pressure may be atmospheric pressure.

Time of reaction may be comprised from 2 to 30 hours, preferably from 5 to 25, more preferably from 10 to 20 hours.

The reaction may be made in presence or an absence of a catalyst. A wide variety of catalysts may be employed with the present reaction. Suitable catalysts include multivalent metal ion salts or organic or inorganic compounds, strong acids and mixtures thereof. Alkali metal oxide catalysts may be used. Examples include zinc oxide, magnesium oxide calcium oxide, zinc sulfate, zinc sulfamate, and zinc oxide acidified with sulfamic or sulfonic acid. Other catalysts which may be used include, but are not limited to, phosphorous based catalysts. Such catalysts include hypophosphorous acidsodium hypophosphite, phosphoric acid, triphosphoric acid, polyphosphoric acid (H₃PO₄), and mixtures thereof.

The level of catalyst may be comprised between 0.1 to 2% by weight based on total reaction mixture on an anhydrous basis, e.g., anhydrous weight of N-methyl taurine plus weight of fatty acid plus weight of catalyst.

Water may be removed during and/or after reaction. During the reaction, the water may be continuously striped off as it is formed. After the reaction, while maintaining the reaction medium hot, vacuum can be applied to help removing residual water and part of the unreacted free fatty acids. Vacuum can be in the range of 900 mbar to 1000 mbar or can also be in a range from 250 mbar to 900 mbar, or can also be in a range from 50 mbar to 250 mbar, or alternatively can also be in the range from 1 mbar to 50 mbar.

Aliphatic taurate amides as produced may be then purified by known purification process such as by evaporation, distillation or filtration.

The evaporation or distillation refers to any suitable separation method for separating two or more components from each other, such as gases from liquid, which separation method is based on utilizing the differences in the vapor pressure of the components.

Examples of such separation methods are evaporation and distillation. The evaporating may be performed in an evaporator using thin film evaporation technology. The evaporator can thus be selected from the group consisting of thin film evaporator, falling film evaporator, short path evaporator and plate molecular still and any other evaporator using thin film technology. The falling film evaporator refers to a falling film tube evaporator. The distillation may be performed in batch or continuous operation modes.

The packing body may be chosen as a function of the efficiency necessary. The packing body can be chosen from the packing bodies well known to a person skilled in the art, such as, for example, solids in the form of rings, polylobal extrudates or saddles. Mention may be made, as nonlimiting examples of packing bodies, of Raschig rings, Pall rings, Intos rings, Berl saddles, Novalox saddles and Intalox saddles. However, the packing body can also be chosen from structured packings.

Structured packing typically consists of thin metal plates that have been arranged in a way to force the fluids to take complicated paths through the column, thereby creating a large surface area for contact between different phases.

Usually the crude aliphatic taurate amide composition comprises about 5 to 10% by weight of free fatty acids; ie. C₆-C₂₄ carboxylic acid having not reacted with the N-methyl taurine.

Aliphatic taurate amide compositions may comprise from 0 to 2% by weight of inorganic salt, with respect to the total weight of the composition.

Aliphatic taurate amide compositions may comprise from 20 to 50% by weight of aliphatic taurate amide, with respect to the total weight of the composition.

Liquid-Liquid Extraction

The present invention refers then to a process of purifying a crude aliphatic taurate amide composition comprising at least the step of proceeding with a liquid/liquid extraction with ethyl acetate of an aqueous composition comprising at least aliphatic taurate amide and C₆-C₂₄ carboxylic acid.

Liquid-liquid extraction, sometimes referred to simply as “liquid extraction” or “solvent extraction’. These interchangeable terms refer to in selectively separating one or more compounds of a mixture on the basis of chemical or physical properties. It concerns the extraction of a substance which is dissolved in a solvent, using another solvent, known as extraction solvent.

The physical principle is the difference in solubility of the product to be extracted between the two liquid phases. Two immiscible fluids or two partially-miscible fluids may be used such that their intimate contact does not yield a single liquid phase.

The aqueous composition comprising an aliphatic taurate amide is preferably an aqueous solution.

The aqueous composition may comprises from 20 to 50% by weight of aliphatic taurate amide, with respect to the total weight of the composition.

The aqueous phase of the aqueous composition is predominantly composed of water, advantageously, it is pure water.

The aqueous composition may comprises from 30 to 80% by weight of water, with respect to the total weight of the composition.

pH of the composition may be comprised from 1 to 13 preferably 2 to 12, more preferably 3 to 10. pH may be chosen in order to keep solubilized the fatty acids in the aqueous medium.

C₆-C₂₄ carboxylic acid is preferably solubilized in the aqueous composition.

Ethyl acetate is present in a quantity sufficient to extract the fatty acids from the aqueous phase.

From 30 to 80% by weight of ethyl acetate, preferably from 40 to 60% by weight of ethyl acetate may be added to the aqueous composition, with respect to the total weight of composition and ethyl acetate.

Other solvents may be added in addition to ethyl acetate, such as for instance alkyl esters, alkyl ethers, alkyl ketons, alkyl alcohols, alkyl aldehydes, aryl esters, aryl ethers, aryl ketons, aryl alcohols, and aryl aldehydes.

The blend may be stirred for a time comprised from 1 minute to 5 hours, notably to allow intimate admixture of the components. Following completion of the agitation step, the mixture is allowed to stand for a sufficient length of time to permit optimum separation of the organic phase comprising fatty acids from the aqueous phase comprising aliphatic taurate amide. This duration will likewise be readily ascertainable by routine experimentation in any particular instance. Following this settling period, the phases may be readily separated from each other in known manner, such as decantation, siphoning, pumping, centrifuging, membrane separation, etc.

It has to be highlighted that no precipitation occurs in the medium further to the addition of ethyl acetate.

The extraction temperature has appeared to be not very critical and can be chosen in a fairly broad range. Temperature during the liquid-liquid extraction may be comprised from 10 to 60° C., notably 15 to 50° C. Pressure may be atmospheric pressure.

Several successive liquid-liquid extraction may be made, for instance 1, 2, 3, 4 or 5 successive extractions to achieve the desired level of residual free fatty acid in the aqueous phase.

The aqueous phase and the organic phase may be introduced countercurrent wise, for example into a liquid/liquid extractor operating continuously. The ratio of the flow rate of the organic phase to the flow rate of the aqueous phase at the start of the liquid/liquid extraction stage is between 2 and 10, preferably between 4 and 8. This ratio is optimized in order to reduce the overall energy consumption.

The number of theoretical stages of the liquid/liquid extractor may be also optimized, so as to reduce by at least 80% by weight, preferably by at least 90% by weight and in particular by at least 95% by weight, the amount of free fatty acids present in the aqueous phase which is subjected to the liquid/liquid extraction operation.

The phase comprising predominantly free fatty acids can advantageously be recycled for at the reaction step after having removed by evaporation the solvent.

Further to the liquid-liquid extraction, it is possible to remove residual ethyl acetate in the aqueous phase notably by stripping treatment or distillation.

Preferred technique to remove the residual ethyl acetate in the liquid phase is by distillation at a temperature between 80 and 120° C., preferably between 95 and 110° C. and more preferably between 99 and 105° C.

Nitrogen or steam can be used to improve ethyl acetate removal by stripping.

The present invention also refers to a process of purifying a crude aliphatic taurate amide composition comprising at least the step of:
(i) proceeding with a liquid/liquid extraction with ethyl acetate of an aqueous composition comprising at least aliphatic taurate amide and C₆-C₂₄ carboxylic acid in order to obtain an aqueous phase comprising aliphatic taurate amide and ethyl acetate, and an organic phase comprising ethyl acetate and C₆-C₂₄ carboxylic acid;
(ii) collect the aqueous phase comprising aliphatic taurate amide and ethyl acetate; and
(iii) remove the ethyl acetate from the aqueous phase comprising aliphatic taurate amide and ethyl acetate, notably by distillation or stripping in order to obtain aqueous phase comprising aliphatic taurate amide.

Aliphatic taurate amide are preferably obtained by reaction of a C₆-C₂₄ carboxylic acid with alkali metal salt of N-methyl taurine.

Aliphatic taurate amide as obtained may be used as solid taurate, notably after drying, or in an aqueous solution with addition of water. Aliphatic taurate amide may suitably be dried to a water content 0 to 10% by weight, preferably 0 to 5% by weight and more preferably 0 to 2% by weight, with respect to the weight of total dried product.

Aliphatic taurate amide as obtained may be used in a liquid composition, notably by addition of water.

Composition

Composition as obtained further to the liquid-liquid extraction may be compositions as follows.

Composition according to the present invention may comprises at least:
a) aliphatic taurate amide, preferably alkyl taurate amide;
b) C₆-C₂₄ carboxylic acid; and
c) ethyl acetate.

Aliphatic taurate amide are preferably obtained by reaction of a C₆-C₂₄ carboxylic acid with alkali metal salt of N-methyl taurine.

Composition according to the present invention may comprises at least:
a) aliphatic taurate amide, preferably alkyl taurate amide; and
b) impurities consisting of C₆-C₂₄ carboxylic acid and ethyl acetate.

Said composition may be liquid or solid.

Liquid composition preferably comprises at least:
a) from 10 to 50% by weight of aliphatic taurate amide, preferably alkyl taurate amide;
b) from 0 to 2% by weight of C₆-C₂₄ carboxylic acid;
c) from 0 to 2% by weight of inorganic salt;
d) from 0 to 2% by weight of alkali metal salt of N-methyl taurine;
e) from 0 to 0.2% by weight of ethyl acetate; and
f) water;
the proportion by weight are calculated with respect to the total weight of the composition.

In the liquid composition, aliphatic taurate amide is comprised from 10 to 50% by weight, preferably from 20 to 50% by weight, more preferably from 20 to 45% by weight.

Said liquid composition comprises from 0 to 2% by weight of C₆-C₂₄ carboxylic acid, preferably from 0 to 1% by weight, more preferably from 0 to 0.5% by weight. Notably said composition can comprises 0, 0.2, 0.4, 0.6, 0.8, 1, 1.2, 1.4, 1.6, 1.8, and 2% by weight of C₆-C₂₄ carboxylic acid, or any possible range constituted by these numbers.

Said liquid composition comprises from 0 to 0.2% by weight of ethyl acetate, preferably from 0 to 0.1% by weight, more preferably from 0 to 0.05% by weight. Notably said composition can comprises 0, 0.01, 0.02, 0.03, 0.04, 0.05, 0.06, 0.07, 0.08, 0.09 and 0.1% by weight of ethyl acetate, or any possible range constituted by these numbers.

Determining components and their amount in the liquid composition may be made for instance as follows:
content of aliphatic taurate amide by NMR
content of C₆-C₂₄ carboxylic acid by NMR and/or by titration using calibrated sodium hydroxide solution
content of inorganic salt (sodium chloride, potassium chloride or any alkali metal chloride) by titration by calibrated silver nitrate solution
content of alkali metal salt of N-methyl taurine by NMR
content of water using Karl Fisher titration

It is also possible to determine components and their amount in the liquid composition for instance as follows:
content of C₆-C₂₄ carboxylic acid by NMR and/or titration using calibrated sodium hydroxide solution
content of inorganic (sodium chloride, potassium chloride or any alkali metal chloride) by titration by calibrated silver nitrate solution
content of alkali metal salt of N-methyl taurine by NM R
content of water using Karl Fisher titration
content of aliphatic taurate amide is then determined by calculation with respect to the amount of other components (100% by weight−% by weight of C₆-C₂₄ carboxylic acid−% by weight of inorganic salt−% by weight of alkali metal salt of N-methyl taurine−% by weight of water).

Preferably the liquid composition of the present invention is consisting of:
a) from 10 to 50% by weight of aliphatic taurate amide, preferably alkyl taurate amide;
b) from 0 to 2% by weight of C₆-C₂₄ carboxylic acid;
c) from 0 to 2% by weight of inorganic salt;
d) from 0 to 2% by weight of alkali metal salt of N-methyl taurine;
e) from 0 to 0.2% by weight of ethyl acetate; and
f) water
the proportion by weight are calculated with respect to the total weight of the composition.

Solid composition according to the present invention preferably comprises at least:
a) from 80 to 99% by weight of aliphatic taurate amide, preferably alkyl taurate amide;
b) from 0 to 5% by weight of C₆-C₂₄ carboxylic acid;
c) from 0 to 2% by weight of inorganic salt;
d) from 0 to 2% by weight of alkali metal salt of N-methyl taurine;
e) from 0 to 0.1% by weight of ethyl acetate; and
f) from 0 to 10% by weight of water;
the proportion by weight are calculated with respect to the total weight of the composition.

In the solid composition, aliphatic taurate amide is comprised from 80 to 99% by weight, preferably from 90 to 99% by weight, more preferably from 95 to 99% by weight.

Said solid composition comprises from 0 to 5% by weight of C₆-C₂₄ carboxylic acid, preferably from 0 to 4% by weight, more preferably from 0 to 3% by weight. Notably said composition can comprises 0, 0.2, 0.4, 0.6, 0.8, 1, 1.2, 1.4, 1.6, 1.8, 2, 2.2, 2.4, 2.6, 2.8, 3, 3.2, 3.4, 3.6, 3.8, 4, 4.2, 4.4, 4.6, 4.8 and 5% by weight of C₆-C₂₄ carboxylic acid, or any possible range constituted by these numbers.

Said solid composition comprises from 0 to 0.1% by weight of ethyl acetate, preferably from 0 to 0.01% by weight, more preferably from 0 to 0,001% by weight. Notably said composition can comprises 0, 0.01, 0.02, 0.03, 0.04, 0.05, 0.06, 0.07, 0.08, 0.09 and 0.1% by weight of ethyl acetate, or any possible range constituted by these numbers.

Preferably the solid composition of the present invention is consisting of:
a) from 80 to 99% by weight of aliphatic taurate amide, preferably alkyl taurate amide;
b) from 0 to 5% by weight of C₆-C₂₄ carboxylic acid;
c) from 0 to 2% by weight of inorganic salt;
d) from 0 to 2% by weight of alkali metal salt of N-methyl taurine;
e) from 0 to 0.1% by weight of ethyl acetate; and
f) from 0 to 10% by weight of water;
the proportion by weight are calculated with respect to the total weight of the composition.

Determining the composition of the solid may be obtained by dissolving the solid in water and apply same methodology as for the liquid technique.

Applications

Aliphatic taurate amide compositions obtained by this treatment are valuable anionic surface active agents and have many varied commercial uses. The most conspicuous property of these products is their great activity at surfaces and interfaces which promotes their use in a large field of the technical arts. For instance, they can be. used as wetting, frothing, or washing agents in the treating and processing of textiles; for converting liquid or solid substances which per so are insoluble in water (such as hydrocarbons, higher alcohols, oils, fats, waxes, and resins) into creamy emulsions, clear solutions or fine stable dispersions; for carbonizing, for dyeing; for the pasting of dyestuffs; for fulling, sizing, impregnating and bleaching treatments; as cleansing agents in hard water; in tanning and mordanting processes; for dyeing acetate with insoluble dyestuffs; for the preparation of dyestuffs in finely divided form; for dispersible dye powders; for producing foam for fire extinguishers; as a means for improving the absorptive power of fibrous bodies; and as an aid in softening hides and skins.

In addition, aliphatic taurate amide compositions products are valuable emulsifiers, wetting agents and dispersants for agricultural compositions containing insecticides, fungicides, bactericides, or other pesticidal substances, herbicides, plant growth regulators, fertilizers and/or soil conditioners, or the like, or mixtures thereof, in solid or liquid form.

These aliphatic taurate amide compositions are also valuable for use as additives to petroleum products, such as fuel oils, lubricating oils, greases, and as additives to the water or brine used for oil. recovery from oil-bearing strata by flooding techniques.

Other valuable uses are in metal cleaning compositions; dry cleaning compositions; additives for rubber lattices; foam inhibitors for synthetic rubber latex emulsions; froth flotation agents; additives for road building materials; as air entraining agents for concrete or cement; additives to asphalt compositions; plasticizers and modifiers for vinyl plastics, alkyl resins, phenolformaldehyde resins and other types of polymeric-type plastic materials; for incorporation into adhesives, paint, linoleum; for use in bonding agents used in various insulating and building materials; as refining aids in wood digesters to prepare pulp, as additives to pulp slurries in beating operations to prevent foaming and also to aid the heating operation in paper-making; and as aids in the preparation of viscose dope.

The products are also useful as emulsifiers for emulsion polymerization, as mercerizing assistants, wetting agents, rewetting agents, dispersing agents, detergents, penetrating agents, softening agents, lime soaps dispersants, dishwashing agents, anti-static agents, disinfectants, insecticides, moth-proofing agents, bactericides, fungicides and biocides. They are valuable as anti-fogging agents for use on glass and other surfaces where the accumulation of an aqueous fog is detrimental. They are of value in hydraulic fluids to improve viscosity characteristics.

Aliphatic taurate amide compositions are especially useful in breaking petroleum emulsions. They may be used to break emulsions of crude petroleum and salt water as obtained from oil wells, or to prevent water-in-oil emulsions resulting from acidization of oil wells by introducing the agent into the well, or to break or prevent emulsions which would result from a water flooding process for recovering oil from oil-bearing strata. They may also be used to break emulsions encountered in a petroleum refining process.

They are useful as corrosion inhibitors, as rust inhibitors, in the protection of metals especially ferrous metals, in acid pickling baths, in acid cleaning compositions, and in electro-plating baths. Other valuable uses are as solvents or in solvent compositions, as cleaning agents for paint brushes, as additives for paints, lacquers, and varnishes; as lubricants, as greases and stufling agents.

Aliphatic taurate amide compositions may be employed in the preparation of homecare compositions such as detergent composition or household cleaner, and also in the preparation of personal care compositions, such as for instance skin creams, lotions, salves and other cosmetic preparations, notably home hair-wave sets, shaving creams, shampoos, toothpastes, etc.

They may also be employed in food products as foaming agents, emulsifying agents, and softening agents.

They may be used as aids in conditioning of soil; as aids in the grinding, milling or cutting of metals either in aqueous solution, emulsions or in oils; as aids in the fixing of dyes to leather and natural or synthetic fibers; as aids in level dyeing of fibers; as aids in stimulating plant growth; as an additive to cement to improve the strength of the resulting concrete or to improve its hardening time or its resistance to freezing and thawing or scaling; and as curing aids and penetrants for use in fertilizer.

### EXPERIMENTAL PART

The invention will now be further illustrated by the following non-limiting examples.

### Example 1: Preparation of Low Free Fatty Acid Liquid Methyl Cocoyl Taurate

a) Crude Methyl Cocoyl Taurate Preparation

378.5 grams (2 moles based) of Coconut Fatty Acid were charged into a 3 neck vessel equipped with 4 pitched blade impeller agitator, distillation column, condenser and decanter. The vessel has been heated to 200° C. 402.4 grams of sodium salt of N-Methyl Taurine aqueous solution (36.5% purity, 1 mole based) was then preheated and charged to the vessel continuously. Water was distilled off into the condenser and collected in the decanter.

When the N-Methyl Taurine charge has been complete, the batch was heated to 238° C. and held for 1 hour as distillate was collected in the decanter. At the reaction temperature, coconut fatty acid formed an amide with the N-Methyl Taurine and by-product water. After the reaction was complete, excess coconut fatty acid was removed from the batch by distillation under vacuum. The molten intermediate has been then converted into liquid methyl cocoyl taurate by dispersing it into water in a 2 liter Flask. The dispersion was then cooled down at 40° C. and adjusted for solid and pH; using water for active, sodium hydroxide accordingly for pH adjustment.

The final methyl cocoyl taurate liquid solution (crude methyl cocoyl taurate) has the following composition by weight percent:
Methyl cocoyl Taurate: 36%
Residual coconut fatty acid: 6%
Residual n-methyl Taurune: not detected
Water: 58%

Methyl cocoyl taurate and n-methyl taurine level have been measured by NIR. Residual free fatty acid were determined by titration using sodium hydroxide as a titrant.

b) Removal of Free Fatty Acid by Liquid-Liquid Extraction Using Ethyl Acetate.

First Liquid/Liquid Extraction Stage

398.6 grams of crude methyl cocoyl taurate was introduced in a 1 liter flask equipped with high speed agitator using 4 pitched blade impeller. Ethyl acetate (394.1 grams, 1:1 ratio) was added into the vessel and the biphasic mixture was stirred at 1100 rpm and heated at 40° C.

After 30 minutes, the mixture has been allowed to settle and the mixture separated in two phases. The lower aqueous phase (462.8 grams) was collected (withdrawn by gravity) with the following composition by weight percent (Liquid Extract 1):
Methyl cocoyl Taurate: 46.97%
Ethyl Acetate: 20.74%
Water: 30.25%
Residual coconut fatty acid: 2.05%

Second Liquid/Liquid Extraction Stage

462.8 grams of “Liquid Extract 1” was re-introduced in the 1 liter. Ethyl acetate (381.7 grams, 1.2:1) was added and same procedure applied for liquid/liquid extraction.

After 30 minutes, the mixture has been allowed to settle and the mixture separated in two phases. The lower aqueous phase (411.7 grams) was collected with the following composition by weight percent (“Liquid Extract 2”):
Methyl cocoyl Taurate: 30.54%
Ethyl Acetate: 23.45%
Water: 44.82%
Residual coconut fatty acid: 1.19%

Third Liquid/Liquid Extraction Stage

411.7 grams of “Liquid Extract 2” was re-introduced in the 1 liter. Ethyl acetate (370.8 grams, 1.1:1) was added and same procedure applied for liquid/liquid extraction.

After 30 minutes, the mixture has been allowed to settle and the mixture separated in two phases. The lower aqueous phase (378.6 grams) was collected with the following composition by weight percent (“Liquid Extract 3”):
Methyl cocoyl Taurate: 29.85%
Ethyl Acetate: 28.25%
Water: 41.08%
Residual coconut fatty acid: 0.82%

c) Finishing Step: Ethyl Acetate Removal from “Liquid Extract 3”

Stripping of “Liquid Extract 3” with two targets:
Removal of Ethyl Acetate from aqueous phase
Adjust active content for the finished methyl cocoyl taurate

To remove Ethyl Acetate in aqueous phase, moderated temperature and pressure stripping conditions have been applied.

Stripping was performed in a jacketed 1-liter flask with temperature control, equipped with a distillation column, a condenser and decanter. 378 grams of “Liquid Extract 3” was then charged to the vessel. Water and Ethyl acetate have been distilled-off into the condenser and collected in the decanter. Distillation temperature started at 80° C. and stopped when liquid mixture reached 104° C. Water can be added to compensate the loss by distillation.

The vessel has been allowed to cooled down before active and pH adjustments; using water for active, sodium hydroxide accordingly for pH adjustment.

The Finished Methyl Cocoyl Taurate (“Pure Liquid Cocoyl taurate”) had the following composition by weight percent and purity:
Methyl cocoyl Taurate: 31.25%
Residual coconut fatty acid: 1.02%
Ethyl Acetate: 0,005% (to be confirmed)
Water: 77%

### Comparative Example 1

Various attempts of removal of free fatty acid from the crude methyl cocoyl taurate of Example 1 have been made by liquid-liquid extraction using other solvents.

Acetone, acetonitrile, chloroform, cyclohexane, toluene, n-pentane, butyl-acetate, and n-propyl acetate all led to the production of gels, without then obtaining two separate phases.

THF and cyclohexanol formed only one phase with the methyl cocoyl taurate.

### Example 2: Preparation of Low Free Fatty Acid Liquid Methyl Lauroyl Taurate

a) Crude Methyl Lauroyl Taurate Preparation

495 grams (2 moles based) of Lauric Acid 99.5% purity was charged into a 3 neck vessel equipped with 4 pitched blade impeller agitator, distillation column, condenser and decanter. The vessel was heated to 200° C. 442.6 grams of sodium salt of N-Methyl Taurine aqueous solution (36.5% purity, 1 mole based) was then preheated and charged to the vessel continuously. Water was distilled off into the condenser and collected in the decanter.

When the N-Methyl Taurine charge was complete, the batch has been heated to 238° C. and held for 1 hour as distillate was collected in the decanter. At the reaction temperature, lauric acid formed an amide with the N-Methyl Taurine and by-product water. After the reaction was complete, excess lauric acid was removed from the batch by distillation under vacuum. The molten intermediate was then converted into liquid methyl lauroyl taurate by dispersing it into water in a 2 liter flask. The dispersion was then cooled down at 40° C. and adjusted for solid and pH.

The final methyl lauroyl taurate liquid solution (crude methyl lauroyl taurate) has the following composition by weight percent:
Methyl lauroyl Taurate: 32%
Residual lauric acid: 8.2%
Residual n-methyl Taurine: not detected
Water: 59.8%

b) Removal of Free Fatty Acid by Liquid-Liquid Extraction Using Ethyl Acetate.

First Liquid/Liquid Extraction Stage

585.8 grams of crude methyl lauroyl taurate was introduced in a 2 liter flask equipped with high speed agitator using 4 pitched blade impeller. Ethyl acetate (591 grams, 1:1 ratio) was added into the vessel and the biphasic mixture was stirred at 1100 rpm and heated at 40° C.

After 30 minutes, the mixture has been allowed to settle and the mixture separated in two phases. The lower aqueous phase (673.8 grams) was collected with the following composition by weight percent (“Liquid Extract 4”):
Methyl lauroyl Taurate: 26.88%
Ethyl Acetate: 21.74%
Water: 48.26%
Residual lauric acid: 3.12%

Second Liquid/Liquid Extraction Stage

673.8 grams of “Liquid Extract 4” was re-introduced in the 1 liter. Ethyl acetate (676.1 grams, 1:1) was added and same procedure applied for liquid/liquid extraction.

After 30 minutes, the mixture has been allowed to settle and the mixture separated in two phases. The lower aqueous phase (621.5 grams) was collected with the following composition by weight percent (“Liquid Extract 5”):
Methyl lauroyl Taurate: 28.95%
Ethyl Acetate: 20.86%
Water: 48.55%
Residual lauric acid: 1.64%

Third Liquid/Liquid Extraction Stage

621.5 grams of “Liquid Extract 5” was re-introduced in the 1 liter. Ethyl acetate (652.2 grams, 1:1) was added and same procedure applied for liquid/liquid extraction.

After 30 minutes, the mixture has been allowed to settle and the mixture separated in two phases. The lower aqueous phase (587.5 grams) was collected with the following composition by weight percent (“Liquid Extract 6”):
Methyl lauroyl Taurate: 30.33%
Ethyl Acetate: 20.96%
Water: 47.47%
Residual lauric acid: 1.24%

a) Finishing Step: Ethyl Acetate Removal from Liquid Extract 6

Stripping of Liquid Extract 6 with two targets:
Removal of Ethyl Acetate from aqueous phase
Adjust active content for the finished methyl lauroyl taurate

To remove ethyl acetate in aqueous phase, moderated temperature and pressure stripping conditions have been applied.

Stripping was performed in a jacketed 1-liter flask with temperature control, equipped with a distillation column, a condenser and decanter. 585 grams of “Liquid Extract 6” was then charged to the vessel. Water and ethyl acetate were distilled-off into the condenser and collected in the decanter. Distillation temperature started at 80° C. and stopped when liquid mixture reached 104° C. Water can be added to compensate the loss by distillation.

The vessel has been allowed to cooled down before active and pH adjustments.

The Finished Methyl Lauroyl Taurate (“Pure Liquid Lauroyl taurate”) had the following composition and purity by weight percent:
Methyl lauroyl Taurate: 32.0%
Residual lauric acid: 1.4%
Ethyl acetate: 0.02%
Water: 66%

### Example 3: Preparation of Low Free Fatty Acid Sodium Methyl Cocoyl Taurate Solid/Powder Form

A solid form of sodium methyl cocoyl taurate has been be obtained by drying of the pure liquid form. A drying step by spray drying has been selected to dry “Pure Liquid Cocoyl taurate” obtained in Example 1.

Nitrogen flow was used to help drying and to have inert drying set-up. The entry temperature of the spray dryer has been set to 235° C., the output temperature set to 110° C. The injector was a bivalve nozzle.

Very pure sodium methyl cocoyl taurate powder was obtained with the following characteristics by weight percent:
Methyl lauroyl Taurate: 95.1%
Residual cocoyl acid: 4.2%
Moisture: 1.2%
Ethyl acetate: 0,0001%

1-20: (canceled)

21. A process of purifying a crude aliphatic taurate amide composition comprising at least the step of proceeding with a liquid/liquid extraction with ethyl acetate of an aqueous composition comprising at least aliphatic taurate amide and C₆-C₂₄ carboxylic acid.

22. The process according to claim 21, wherein the aliphatic taurate amide is obtained by reaction of a C₆-C₂₄ carboxylic acid with an alkali metal salt of N-methyl taurine.

23. The process according to claim 21, wherein the aliphatic taurate amides are alkyl taurate amides or alkenyl taurate amides.

24. The process according to claim 21, wherein the aliphatic taurate amide is selected from sodium methyl lauroyl taurate, sodium methyl ceteoyl taurate, sodium methyl palmitoyl taurate, sodium methyl oleyl taurate, sodium methyl stearoyl taurate, sodium methyl cocoyl taurate, and mixtures thereof.

25. The process according to claim 21, wherein the C₆-C₂₄ carboxylic acid is selected from caprylic acid, octanoic acid, decanoic acid, lauric acid, cocoyl acid, tridecylic acid, myristic acid, pentadecylic acid, palmitic acid, margaric acid, stearic acid, nonadecylic acid, arachidic acid, behenic acid, pyroterebic acid (4-methyl-β-pentenoic acid), α-ethylcrotonic acid, teracrylic acid, d-citronellic acid, θ-undecylenic acid, oleic acid, elaidic acid, erucic acid, sorbic acid, stearolic acid, linoleic acid, behenoleic acid, ricinoleic acid, margaric acid, arachidic acid, and mixtures thereof.

26. The process according to claim 21, wherein the aqueous composition comprising the aliphatic taurate amide is an aqueous solution.

27. The process according to claim 21, wherein the aqueous composition comprises from 20% to 50% by weight of the aliphatic taurate amide, with respect to the total weight of the composition.

28. The process according to claim 21, wherein the C₆-C₂₄ carboxylic acid is solubilized in the aqueous composition.

29. The process according to claim 21, wherein from 30% to 80% by weight of ethyl acetate is added to the aqueous composition, with respect to the total weight of composition and ethyl acetate.

30. The process according to claim 21, wherein the temperature during the liquid-liquid extraction ranges from 10° C. to 60° C.

31. The process according to claim 21, wherein the ethyl acetate added to the liquid-liquid extraction is removed by stripping treatment or distillation.

32. A process of purifying a crude aliphatic taurate amide composition comprising at least the step of:
(i) proceeding with a liquid/liquid extraction with ethyl acetate of an aqueous composition comprising at least aliphatic taurate amide and C₆-C₂₄ carboxylic acid in order to obtain an aqueous phase comprising the aliphatic taurate amide and the ethyl acetate, and an organic phase comprising the ethyl acetate and the C₆-C₂₄ carboxylic acid;
(ii) collecting the aqueous phase comprising the aliphatic taurate amide and the ethyl acetate; and
(iii) removing the ethyl acetate from the aqueous phase comprising the aliphatic taurate amide and the ethyl acetate.

33. The process according to claim 32, wherein the aliphatic taurate amide is obtained by reaction of a C₆-C₂₄ carboxylic acid with an alkali metal salt of N-methyl taurine.

34. A purified product susceptible to be obtained according to the process according to claim 21.

35. The process according to claim 21, wherein the ethyl acetate is a solvent for the liquid/liquid extraction.

36. A composition comprising at least:
a) an aliphatic taurate amide;
b) a C₆-C₂₄ carboxylic acid; and
c) ethyl acetate.

37. The composition according to claim 36, wherein the aliphatic taurate amide is obtained by reaction of a C₆-C₂₄ carboxylic acid with an alkali metal salt of N-methyl taurine.

38. A liquid composition comprising at least:
a) from 10% to 50% by weight of an aliphatic taurate amide;
b) from 0% to 2% by weight of a C₆-C₂₄ carboxylic acid;
c) from 0% to 2% by weight of an inorganic salt;
d) from 0% to 2% by weight of an alkali metal salt of N-methyl taurine;
e) from 0% to 0.2% by weight of ethyl acetate; and
f) water
the proportion by weight are calculated with respect to the total weight of the composition.

39. A solid composition comprising at least:
a) from 80% to 99% by weight of an aliphatic taurate amide;
b) from 0% to 5% by weight of a C₆-C₂₄ carboxylic acid;
c) from 0% to 2% by weight of an inorganic salt;
d) from 0% to 2% by weight of an alkali metal salt of N-methyl taurine;
e) from 0% to 0.1% by weight of ethyl acetate; and
f) from 0% to 10% by weight of water;
the proportion by weight are calculated with respect to the total weight of the composition.

40. The process according to claim 29, wherein from 40% to 60% by weight of ethyl acetate is added to the aqueous composition, with respect to the total weight of composition and ethyl acetate.
